(19) United States
(12) Patent Application Publication
(10) Pub. No.: US 2016/0058631 A1
(43) Pub. Date: Mar. 3, 2016

(54) CLOTH DIAPER WITH DUAL-CLOSURE SYSTEM

(71) Applicant: The Natural Baby Company LLC, Bozeman, MT (US)

(72) Inventor: Kim Ormsby, Bozeman, MT (US)

(21) Appl. No.: 14/831,553

(22) Filed: Aug. 20, 2015

Related U.S. Application Data
(60) Provisional application No. 62/045,152, filed on Sep. 3, 2014.

(51) Int. Cl. A61F 13/56; A61F 13/62

(52) U.S. Cl. CPC A61F 13/5644; A61F 13/56; A61F 13/622; A61F 2013/5694; A61F 2013/5677

(57) ABSTRACT

A cloth diaper comprising a from panel, a plurality of snaps configured in a first row situated directly underneath the top edge of the from panel, a strip of hook-and-loop fastener material that snaps onto the plurality of snaps configured in a first row and situated directly underneath the top edge of the front panel, a rear panel, a first side wing comprising at least one snap on an inside surface of the first side wing, a second side wing comprising at least one snap on an inside surface of the second side wing, a first side wing hook-and-loop attachment member that snaps onto the at least one snap on the inside surface of the first side wing, and a second side wing hook-and-loop attachment member that snaps onto the at least one snap on the inside surface of the second side wing.

### CROSS-REFERENCE TO RELATED APPLICATION

Pursuant to 35U.S.C. §119(e), this application claims priority back to U.S. Patent Application No. 62/045,152 filed on Sep. 3, 2014.

### BACKGROUND OF THE INVENTION

1. Field of the Invention

The present invention relates generally to the field of diapers, and more particularly, to a cloth diaper with a dual-closure system.

2. Description of the Related Art

There have been a number of innovations to closure systems for cloth diapers but none that provides both a snap closure system and a hook-and-loop fastener closure system. Snaps have been used on diapers for size adjustment (that is, along the front of the diaper), and they have also been used as closure systems (wherein the side wings snap onto the front panel of the diaper). Hook-and-loop fasteners have been used as closure systems wherein the side wings fold over onto the front panel and are held in place by hook-and-loop fasteners. No diaper, however, has combined both a snap closure system and a hook-and-loop fastener system, allowing the consumer to choose between the two closure systems. Furthermore, the hook-and-loop fastener system of the present invention is replaceable.

U.S. Pat. No. 4,892,598 (Stevens et al., 1990) discloses a method of integrating a fluid-absorbing component into a holding component of an anatomically form-fitting absorbent garment. The garment comprises a slidable hem retaining arrangement that connects a relatively inelastic absorbent insert to the outer cover. The side wings (which the inventor calls “ear portions”) include both snap and hook-and-loop fasteners, but these systems are not interchangeable, and they are not situated across the from of the top panel of the diaper so as to provide unlimited sizing options.

U.S. Pat. No. 4,923,456 (Proxmire, 1990) provides a disposable diaper with an absorbent structure integrated within an outer cover. The side wings (or “ear portions”) are shown with both snap and hook-and-loop fasteners, but these fastening systems are not interchangeable, the hook-and-loop fastening system is not replaceable, and the closure systems do not extend across the entire top portion of the front panel of the diaper, as in the present invention.

U.S. Pat. No. 4,981,480 (Gaudet et al., 1991) discloses an adjustable child garment that can be worn as a diaper or a diaper wrap from birth until diapers are no longer required. The garment comprises a hook-and-loop fastening system that extends across the top portion of the front panel of the garment. In an alternate embodiment, a snap closure system is used in lieu of the hook-and-loop fastener. The invention does not contemplate providing both types of fasteners on the same garment.

U.S. Pat. No. 5,019,068 (Perez et al., 1991) provides a washable diaper with an absorbent liner that is folded within the diaper during use. An additional liner is optionally secured to the first liner where extra absorbency is required. The only fastening system shown is a hook-and-loop fastener.

U.S. Pat. No. 5,423,789 (Kuen, 1995) discloses an absorbent garment with a pair of strap members and a garment shell. The strap members are fastened to the shell with both buttons and hook-and-loop fasteners.

U.S. Pat. No. 8,430,857 (Labit et al., 2013) provides a reusable diaper with corner tabs that are releasably attached to the forward and rearward waist portions of the diaper. According to the specification, the corner tabs may be attached to various portions of the diaper with snaps, buttons, hook-and-loop fasteners or adhesives. The diaper comprises throe rows of snaps across the from panel of the diaper for adjustment of crotch length. In various embodiments, the diaper is shown with 4 snap closure system and a hook-and-loop closure system but never both systems in the same embodiment.

U.S. Pat. No. 8,518,007 (Labit et al., 2013) comprises a reusable diaper with as three-by-three array of snap members on the front panel of the diaper for adjustment of crotch length. The corner regions of the diaper may be releasably attachable to the diaper and/or resiliently stretchable. The diaper is shown only with a hook-and-loop fastening system.

U.S. Pat. No. 8,728,052 (Wang et al., 2014) provides a convertible reusable diaper with a detachable absorption portion. The diaper comprises a snap closure system but does not utilize hook-and-loop fasteners.

U.S. Pat. No. 8,777,915 (Labit et al., 2014) discloses a reusable diaper with seam allowances within the corner regions to help inhibit wicking of liquid. The diaper comprises a hook-and-loop closure system and a snap system for adjustment of diaper size (crotch length). The invention does not integrate both a hook-and-loop and a snap closure system.

U.S. Pat. No. 8,992,498 (Labit et al., 2015) provides a reusable diaper with a fluid-absorbing insert coupled to one or more inner portions of the diaper for use in absorbing fluids. The fluid-absorbing insert is adjustable to accommodate use by a male or female. The diaper is shown alternately with a hook-and-loop closure system and a snap closure system but not with both closure systems in the same embodiment.

U.S. Pat. No. 8,961,484 (Ekstrom, 2015) discloses a double gusset cloth diaper with a fluid absorbent material that is removably located in a recess in the groin area of the diaper. The preferred embodiment comprises a snap closure system only. In an alternate embodiment, hook-and-loop fasteners are used in lieu of the snaps. The invention does not incorporate both types of fasteners into a single embodiment.

U.S. Patent Application Pub. No. 2002/0111596 (Fletcher et al.) describes a pant-like absorbent garment with removable side panels. The removable side panels are attached to the absorbent chassis with hook-and-loop fasteners. No snaps are used in connection with this invention.

### BRIEF SUMMARY OF THE INVENTION

The present invention is a cloth diaper comprising: a front panel comprising a top edge, a plurality of snaps configured in a first row situated directly underneath the top edge of the front panel, and a strip of hook-and-loop fastener material that snaps into the plurality of snaps configured in a first row and situated directly underneath the top edge of the front panel; a rear panel; a first side wing comprising at least one snap on an inside surface of the first side wing; a second side wing comprising at least one snap on an inside surface of the second side wing; a first side wing hook-and-loop attachment member that snaps onto the at least one snap on the inside surface of the first side wing; and a second side wing hook-and-loop attachment member that snaps onto the at least one snap on the inside surface of the second side wing. In a preferred embodiment, the front panel, the rear panel, the first side wing, and the second side wing are each comprised of a waterproof outer layer and an absorbent inner layer.

In a preferred embodiment, each of the side wing hook-and-loop attachment members is comprised of a first layer of hook-and-loop fastener material and a second layer of hook-and-loop material, wherein each of the side wing hook-and-loop attachment members comprises at least one snap socket, each having a female portion and a flat top, wherein the female portion of the snap sockets extends through a first layer of hook-and-loop material, and wherein the flat top of the snap socket is situated in between the first and second layers of hook-and-loop material. Preferably, the invention further comprises two removable absorbent liners.

In a preferred embodiment, the front panel further comprises three rows of snaps situated directly underneath the first row of snaps. Preferably, the first side wing comprises a patch of hook-and-loop material situated on an outside surface of the first side wing and permanently stitched to the first side wing. Preferably, the at least one snap on the second side wing is a snap cover having a male part that faces an inner region of the diaper, and the second side wing further comprises a snap socket having a female portion situated on an outer surface of the second side wing.

### BRIEF DESCRIPTION OF THE DRAWINGS

FIG. 1 is a perspective view of the present invention shown with the diaper closed and the hook-and-loop closure system in place.

FIG. 2 is an inside perspective view of the present invention shown with the diaper open and the hook-and-loop closure system in place.

FIG. 3 is a perspective view of the present invention shown with the hook-and-loop closure system removed.

FIG. 4 is an inside perspective view of the present invention shown with the diaper open and the hook-and-loop closure system removed.

FIG. 5 is an inside perspective view of the present invention shown with the diaper open and the hook-and-loop closure system and absorbent liners removed.

FIG. 6 is an outside perspective view of the present invention shown with the diaper open and the hook-and-loop closure system removed.

FIG. 7 is an inside perspective view of the present invention shown with the diaper open and the hook-and-loop closure system in place with the two absorbent liners removed.

FIG. 8 is an inside perspective view of the present invention shown with the diaper open, the hook-and-loop closure system in place, the first of the two absorbent liners removed.

FIG. 9 is a perspective view of the present invention shown in a display or storage state with the hook-and-loop fastener system in place.

FIG. 10 is a perspective view of the present invention shown in a display or storage state with the hook-and-loop fastener system removed.

### REFERENCE NUMBERS

1 Front panel

1a Snap socket

1b Snap cover

1c Snap socket

1d Snap socket

2 Rear panel

3 First side wing

3a Snap cover

4 Second side wing

4a Snap cover

5 Border of waterproof material

6 Strip of hook-and-loop fastener material

6a Snap cover

7 Side wing hook-and-loop attachment member

7a Snap socket

8 First absorbent liner

8a Snap over

8b Snap socket

9 Second absorbent liner

9a Snap cover

10 Snap socket

11 Patch of hook-and-loop fastener material

12 Snap socket

### DETAILED DESCRIPTION OF INVENTION

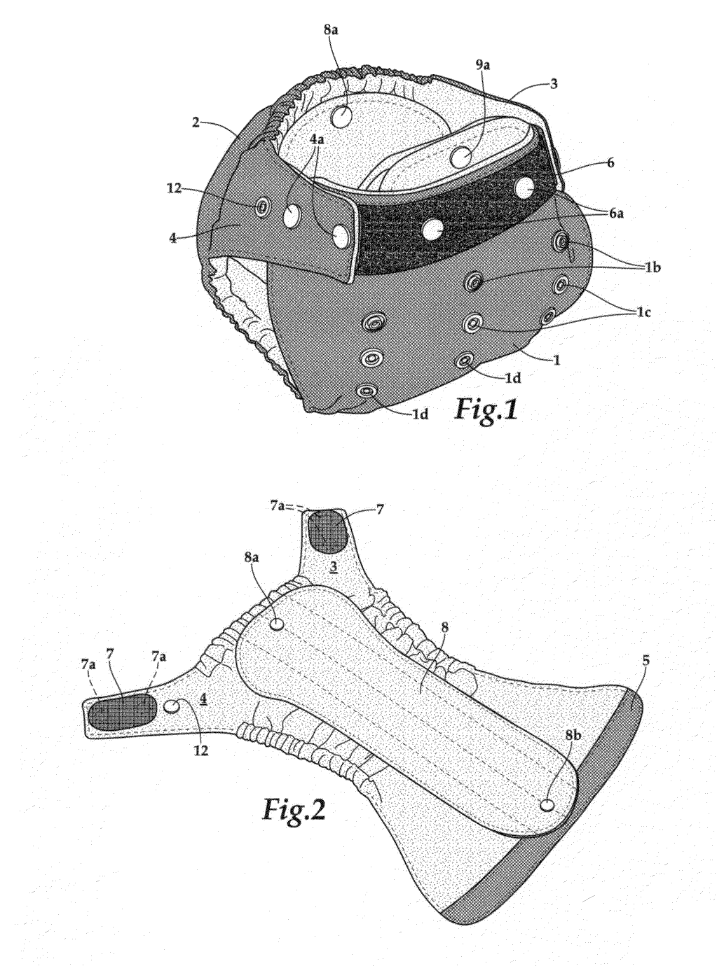

FIG. 1 is a perspective view of the present invention shown with the diaper closed and the hook-and-loop closure system in place. As shown in this figure, the present invention is a diaper comprised of a front panel 1, a rear panel 2, a first side wing 3 and a second side wing 4. The front and rear panels 1, 2 and first and second side wings 3, 4 are preferably comprised of a waterproof outer layer and an absorbent inner layer. In a preferred embodiment, the inner layer of the front and rear panels and first and second side wings is to single layer of organic cotton material. Preferably, the inner layer comprises a border of waterproof material 5 that extends roughly an inch from the top edge of the front panel (as shown in FIG. 2). In a preferred embodiment, the outer layer of the front and rear panels, which join together in the crotch area of the diaper, is a single layer of waterproof material. As such, there is no seam in the crotch area on either the inner or the outer layers. The outer layers of the first and second side wings are preferably sewn (stitched) to the outer layer of the rear panel, as shown in the figures.

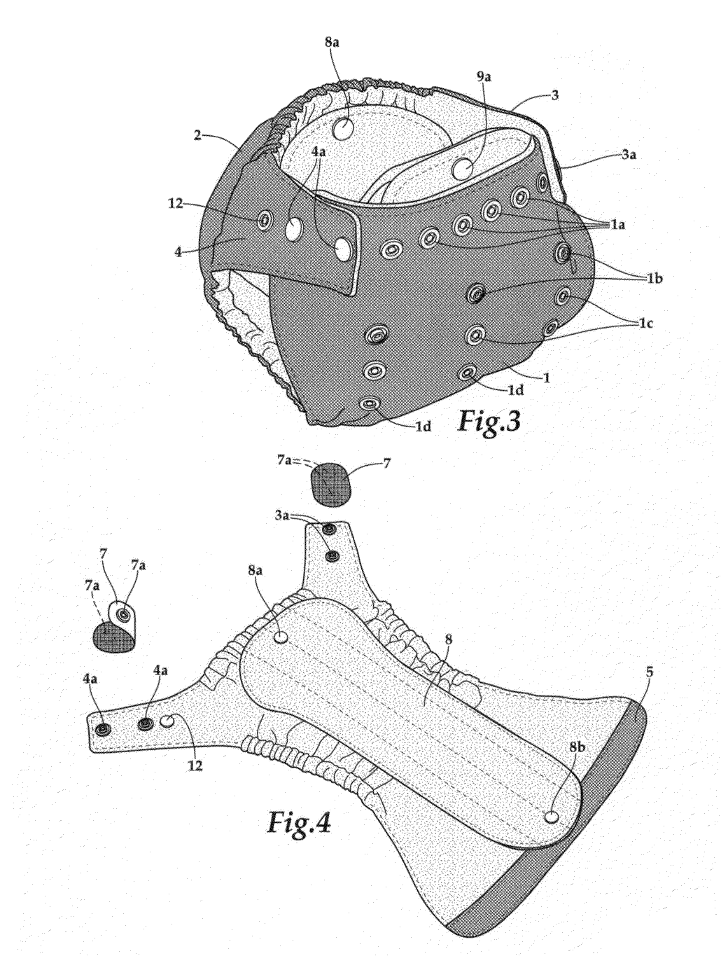

In a preferred embodiment, a strip of hook-and-loop fastener material 6 (comprised of the loop portion only) comprised of a number of snap covers 6a snaps onto snap sockets 1a (see FIG. 3) that are aligned in a single row across the top of the front panel 1 of the diaper (see FIG. 3). Note that the number of snap covers 6a may be less than the number of snap sockets 1a. (For purposes of description, each snap is described as comprised of a snap cover and a snap socket. Each snap cover has a male portion that engages with a female portion of the snap socket.) Each side wing 3, 4 comprises at least one but preferably two snap covers 3a, 4a, the male part of which faces the inner region of the diaper (see FIG. 4). Side wing hook-and-loop attachment members 7 snap onto the snap covers 3a, 4a on the side wings 3, 4. In a preferred embodiment, each of the side wing hook-and-loop attachment members 7 comprises two layers of hook-and-loop fastener material (the first layer being comprised of loop material and the second layer being comprised of hook material) that are sewn together such that the loops of the first layer face outward and the hooks of the second layer face outward, with the female portions of two snap sockets 7a extending through the first layer and the flat tops of the two snap sockets situated in between the two layers (so that they are not visible).

FIG. 3 is a perspective view of the present invention shown with the hook-and-loop closure system removed. In this figure, the strip 6 has been unsnapped from the top of the front panel 1 of the diaper, and the side wing hook-and-loop attachment members 7 have also been unsnapped from the side wings 3, 4. To close the diaper using the snap system only, the side wings 3, 4 are wrapped around the side of the child, and the snap covers 3a, 4a in the side wings 3, 4 are snapped into any of the snap sockets 1a in the row along the top of the front panel 1 of the diaper. The dual-closure system of the present invention is preferred over other diapers because it allows the consumer to elect not to use the hook-and-loop closure system or to replace if and when it becomes worn.

FIG. 4 is an inside perspective view of the present invention shown with the diaper open and the hook-and-loop closure system removed. As shown in this figure, the side wing hook-and-loop attachment members 7 can be removed by simply unsnapping them from the snap sockets in the side wings 3, 4.

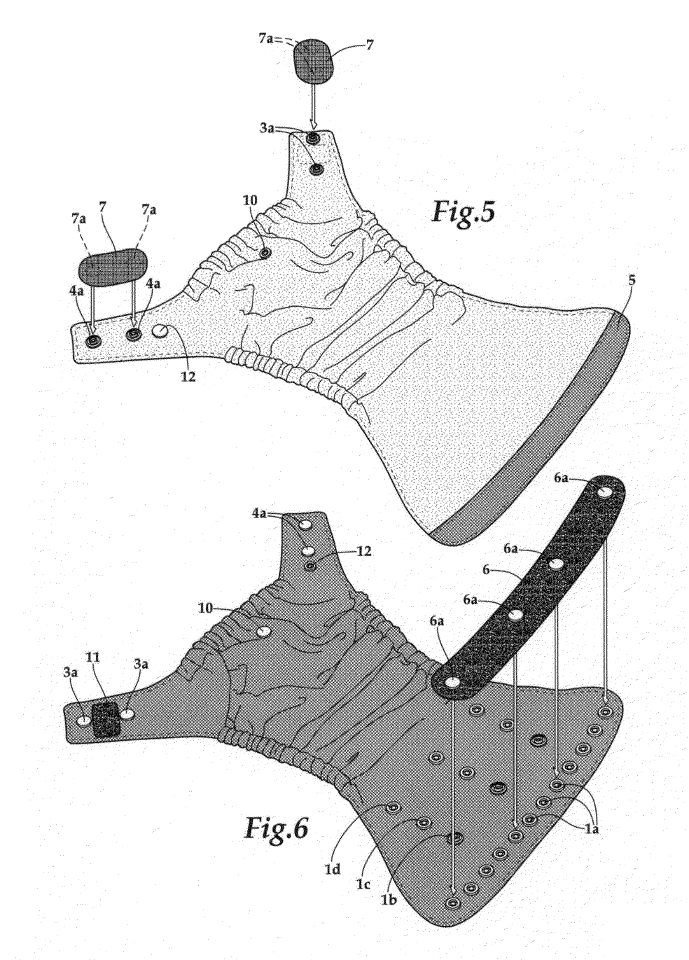

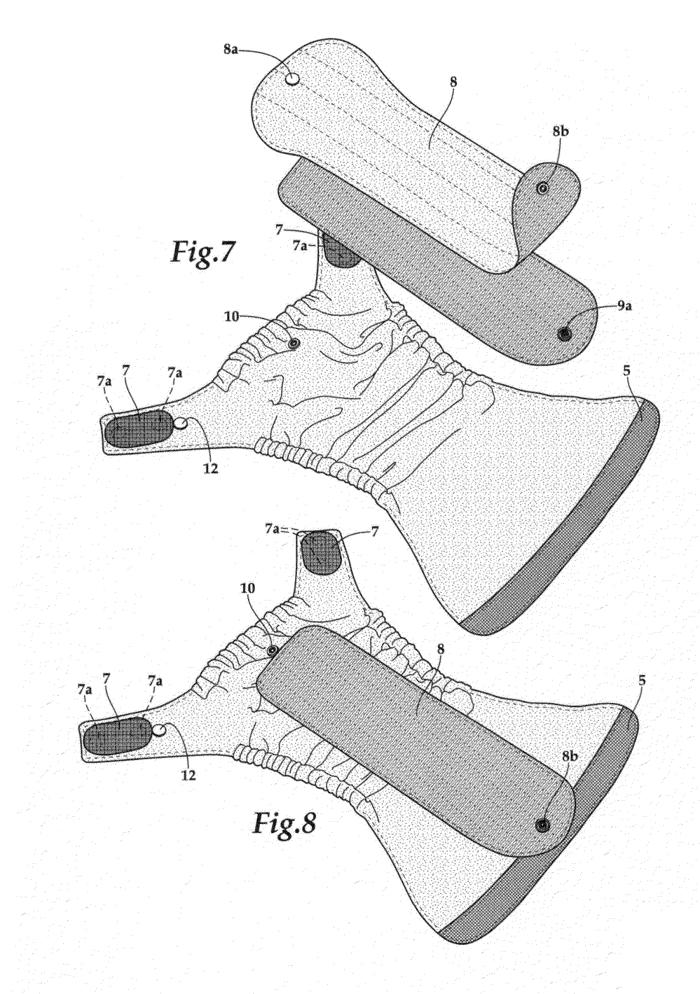

FIG. 5 is an inside perspective view of the present invention shown with the diaper open and the hook-and-loop closure system and absorbent liners removed. In a preferred embodiment, the invention comprises two absorbent liners that snap into the inside of the diaper for additional absorbency. The configuration of the two liners is discussed in connection with FIG. 7 below.

FIG. 6 is an outside perspective view of the present invention shown with the diaper open and the hook-and-loop closure system removed. As shown in this figure, the strip 5 can be removed from the diaper simply by unsnapping it from the row of snap sockets 1a. In a preferred embodiment, the front panel of the diaper preferably further comprises three rows of snaps situated on the front panel of the diaper underneath the row of snap sockets 1a. The first row of snaps 1b preferably comprises three snap covers with the male portions facing outward. The second row of snaps 1c preferably comprises three snap sockets with the female portions facing outward. The third row of snaps 1d preferably comprises three snap sockets with the female portions facing outward. This particular configuration of snap covers and snap sockets allows the second or third row of snap sockets 1c, 1d to be snapped into the first row of snap covers 1b to shorten the length of the diaper.

FIG. 7 is an inside perspective view of the present invention shown with the diaper open and the hook-and-loop closure system in place with the two absorbent liners removed. In a preferred embodiment, the invention further comprises a first absorbent liner 8 and is second absorbent liner 9. The first absorbent liner 8 snaps onto a snap socket 10 at the top of the diaper on the inside of the rear panel 2 via a snap cover 8a on one end of the liner 8. The second absorbent liner 9 snaps via a snap cover 9a onto a snap socket 8b located on the other end of the first absorbent liner 8. Both liners 8, 9 may be used at the same time for extra absorbency.

FIG. 8 is an inside perspective view of the present invention shown with the diaper open, the hook-and-loop closure system in place, and the first of the two absorbent liners removed. In this figure, only the second absorbent liner 9 is used; the snap cover 9a on the liner 9 snaps into the snap socket 10.

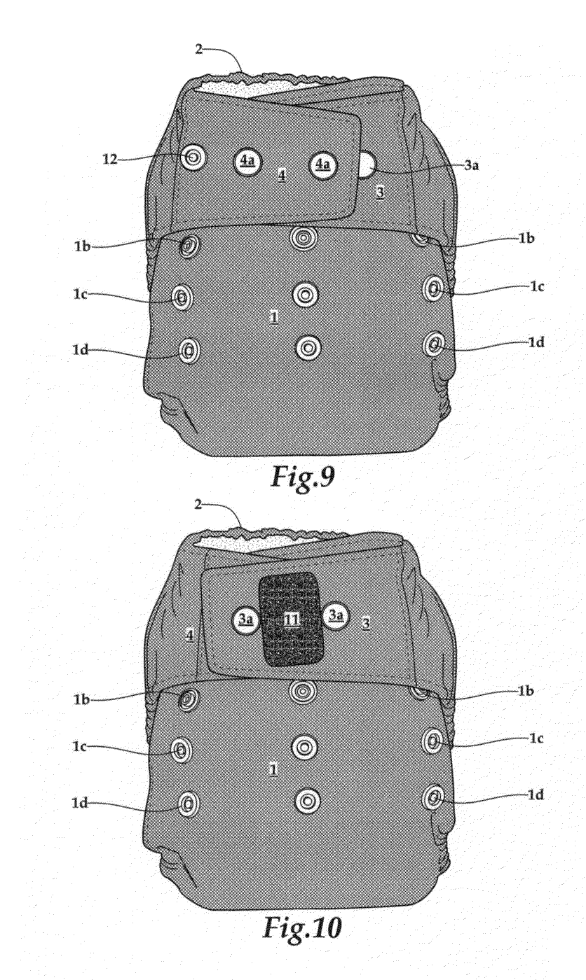

FIG. 9 is a perspective view of the present invention shown in a display or storage state with the hook-and-loop fastener system in place. As shown in this figure, the side wing hook-and-loop fastener 7 on the inside of the left-hand side wing 4 wraps onto the patch of hook-and-loop fastener 11 (loop portion only) that is situated between the two snap covers 3a on the side wing 3. The patch of hook-and-loop fastener material 11 is preferably permanently stitched to the side wing 3.

FIG. 10 is a perspective view of the present invention shown in a display or storage state with the hook-and-loop fastener system removed. As shown in this figure, when the side wing hook-and-loop fasteners 7 are removed, either one of snap covers 3a can snap onto snap socket 12, which is situated on the left-hand side wing 4 just inside of two snap covers 4a. Note that the female portion of the snap socket is located on the outer surface of the side wing 4.

Although the preferred embodiment of the present invention has been shown and described, it will be apparent to those skilled in the art that many changes and modifications may be made without departing from the invention in its broader aspects. The appended claims are therefore intended to cover all such changes and modifications as all within the true spirit and scope of the invention.

I claim:

1. A cloth diaper comprising:
(a) a front panel comprising a top edge, a plurality of snaps configured in a first row situated directly underneath the top edge of the front panel, and a strip of hook-and-loop fastener material that snaps onto the plurality of snaps configured in a first row and situated directly underneath the top edge of the front panel;
(b) a rear panel;
(c) a first side wing comprising at least one snap on an inside surface of the first side wing;
(d) a second side wing comprising at least one snap on an inside surface of the second side wing;
(e) a first side wing hook-and-loop attachment member that snaps onto the at least one snap on the inside surface of the first side wing; and
(f) a second side wing hook-and-loop attachment member that snaps onto the at least one snap on the inside surface of the second side wing.

2. The cloth diaper of claim 1, wherein the front panel, the rear panel, the first side wing, and the second side wing are each comprised of a waterproof outer layer and an absorbent inner layer.

3. The cloth diaper of claim 1, wherein each of the side wing hook-and-loop attachment members is comprised of a first layer of hook-and-loop fastener material and a second layer of hook-and-loop material, wherein each of the side wing hook-and-loop attachment members comprises at least one snap socket, each having a female portion and a flat top, wherein the female portion of the snap sockets extends through a first layer of hook-and-loop material, and wherein the flat top of the snap socket is situated in between the first and second layers of hook-and-loop material.

4. The cloth diaper of claim 1, further comprising two removable absorbent liners.

5. The cloth diaper of claim 1, wherein the front panel further comprises three rows of snaps situated directly underneath the first row of snaps.

6. The cloth diaper of claim 1, wherein the first side wing comprises a patch of hook-and-loop material situated on an outside surface of the first side wing and permanently stitched to the first side wing.

7. The cloth diaper of claim 1, wherein the at least one snap on the second side wing is a snap cover having a male part that faces an inner region of the diaper, and wherein the second side wing further comprises a snap socket having a female portion situated on an outer surface of the second side wing.
